(19) United States
(12) Patent Application Publication
(10) Pub. No.: US 2023/0166128 A1
(43) Pub. Date: Jun. 1, 2023

(54) ULTRASOUND VORTEX ENERGY INJECTION DEVICE

(71) Applicant: Mose Medi Inc., Gumi-Si (KR)

(72) Inventor: Day JOH, Daegu (KR)

(21) Appl. No.: 17/875,848

(22) Filed: Jul. 28, 2022

(30) Foreign Application Priority Data
Nov. 30, 2021 (KR) 10-2021-0167836

(51) Int. Cl. A61N 7/00; B06B 1/06

(52) U.S. Cl. CPC A61N 7/00; B06B 1/0625; B06B 2201/76; A61N 2007/0034; A61N 2007/0078

(57) ABSTRACT

Disclosed is an ultrasound vortex energy injection device configured in such a way as to disperse ultrasound waves without focusing the ultrasound waves with respect to an area coming in contact with a human body, and at the same time, apply a vortex-type stimulus in a circumferential direction of an area getting in contact with the human body with respect to a plurality of piezoelectric elements arranged to be spaced apart from one another in a circumferential direction (clockwise or counterclockwise) of an area opposed to the human body where ultrasound waves are transmitted.

### CROSS-REFERENCE TO RELATED APPLICATION

This application claims priority from Korean Patent Application No. 10-2021-0167836, filed on Nov. 30, 2021, in the Korean Intellectual Property Office, the disclosure of which is incorporated herein by reference in its entirety.

### BACKGROUND OF THE INVENTION

### Field of the Invention

The present invention relates to an ultrasound vortex energy injection device and, more specifically, to an ultrasound vortex energy injection device configured in such a way as to disperse ultrasound waves without focusing the ultrasound waves with respect to an area coming in contact with a human body, and at the same time, apply a vortex-type stimulus in a circumferential direction of an area getting in contact with the human body with respect to a plurality of piezoelectric elements arranged to be spaced apart from one another in a circumferential direction (clockwise or counterclockwise) of an area opposed to the human body where ultrasound waves are transmitted.

### Background Art

In order to remove skin wrinkles or treat cancer, high intensity focused ultrasound (HIFU), low intensity focused ultrasound (LIFU), low intensity focused ultrasound (LFU), and low intensity high pulse ultrasound waves have been widely used.

The high intensity focused ultrasound (HIFU) is a method of focusing ultrasound waves and instantaneously raising the temperature inside the human body up to approximately 70° C. so as to necrotize tissues or cells of a target area. When the heat is focused on the SAMA layer, which corresponds to a target, existing between the subcutaneous layer and the muscle layer, the HIFU reduces fine wrinkles due to collagen denaturation and generates strong heat by the high intensity focused ultrasound waves applied to the tumor corresponding to the target so that the tumor is ablated by using cavitation and waves with respect to the tissues. However, the conventional high intensity focused ultrasound (HIFU) has side effects, such as burn, by focusing and irradiating strong energy to the central portion of the target. In this instance, the heat energy to be irradiated is focused on the central portion of the lesion to generate a heat storage phenomenon. Therefore, a thermal runaway is generated. Due to the thermal island phenomenon in which the central portion is overheated, a patient to be treated cannot withstand excessive burning sensation and thus cannot be treated for a period of time required for treatment.

Such a phenomenon is referred to as a thermal equilibrium state. In this instance, a passage through which heat put inside is discharged out must be prepared, but the conventional method cannot solve the above problem. In order to solve the problem, development of an ultrasound vortex injection device is required. An ultrasound vortex induction device of the ultrasound vortex injection device distributes and circulates the input heat from the inside and outside of piezoelectric elements. In addition, a doughnut-shaped ultrasound transducer disperses and overlaps fine energy to prevent the thermal runaway and break the equilibrium state of heat around the lesion to induce ultrasound vortex, thereby enabling energy to be introduced for a required period of time without excessive thermal sensation.

Moreover, if existing ultrasound waves do not reach a target accurately, due to the risk of damage with respect to normal tissues, only one point at a time can be treated.

Furthermore, the conventional high-intensity focused ultrasound waves have a disadvantage in that it is difficult to move oxygen in the capillary tube around the target due to the thermal equilibrium state around the target, and it causes oxygen deficit.

In order to solve such thermal equilibrium state and the thermal runaway phenomenon with respect to the central portion to which ultrasound waves are irradiated, it is necessary to disperse irradiated waves and heat, and it is also required to develop technology to discharge heat from the piezoelectric elements.

### PATENT LITERATURE

### Patent Documents

Patent Document 1: Korean Patent No. 10-1514401 (granted on Apr. 16, 2015)
Patent Document 2: Japanese Patent Laid-open No. 10-234734 (published on Sep. 8, 1998)

### SUMMARY OF THE INVENTION

Accordingly, the present invention has been made in view of the above-mentioned problems occurring in the prior art, and it is an object of the present invention to provide an ultrasound vortex energy injection device configured in such a way as to disperse ultrasound waves without focusing the ultrasound waves with respect to an area coming in contact with a human body, and at the same time, apply a vortex-type stimulus in a circumferential direction of an area getting in contact with the human body with respect to a plurality of piezoelectric elements arranged to be spaced apart from one another in a circumferential direction (clockwise or counterclockwise) of an area opposed to the human body where ultrasound waves are transmitted.

To accomplish the above object, according to the present invention, there is provided an ultrasound vortex energy injection device, which is configured in such a way as to disperse ultrasound waves without focusing the ultrasound waves with respect to an area coming in contact with a human body, and at the same time, apply a vortex-type stimulus in a circumferential direction of an area getting in contact with the human body with respect to a plurality of piezoelectric elements arranged to be spaced apart from one another in a circumferential direction (clockwise or counterclockwise) of an area opposed to the human body where ultrasound waves are transmitted, the ultrasound vortex energy injection device including: a lower housing, which has a doughnut-shaped ultrasound transducer having a vacuum hole perforated at the center, and a trumpet-shaped vacuum unit protruding and extending upward from the vacuum hole of the ultrasound transducer and having a vacuum groove; three or more piezoelectric elements arranged on the outer circumference of an inclined area which is formed at a lower area of the area in which the vacuum groove of the vacuum unit is formed, and of which the circumference is gradually increased, the three or more piezoelectric elements being arranged on the inclined area at equal intervals; an upper housing connected to the outer circumferential surface of the ultrasound transducer of the lower housing and having a connector insertion hole formed at one side of the outer circumferential surface of the upper end; a connector which has a central portion penetrating the connector insertion hole to communicate with the connector connection hole, and a circumferential portion provided with a cable of a ultrasound generator electrically connected to the piezoelectric elements, the cable being disposed around the outer circumferential surface of the central portion; and a control unit which includes a vacuum generator connected to the central portion of the connector by means of a hose, and a ultrasound generator electrically connected to the cable of the ultrasound generator so as to control the operation of the vacuum generator and the ultrasound generator.

Especially, wherein with respect to a second piezoelectric element, a third piezoelectric element, . . . , and a N^{th} piezoelectric element (wherein N is 1, 2, 3, . . . ) which are arranged to be spaced apart from one another in sequential order in the clockwise direction based on a first piezoelectric element, the control unit controls energy injected using any one among frequency (MHz), pressure (w/cm²), volt (V), and relay time of the ultrasound generator as an independent variable to satisfy the sequential order of the first piezoelectric element, the second piezoelectric element, the third piezoelectric element, . . . , and the N^{th} piezoelectric element, thereby continuously forming counterclockwise ultrasound vortex in sequential order of a body area receiving ultrasound waves from the N^{th} piezoelectric element, . . . , a body area receiving ultrasound waves from the third piezoelectric element, a body area receiving ultrasound waves from the second piezoelectric element, and a body area receiving ultrasound waves from the first piezoelectric element due to a difference of thermal storage energy in proportion to injected energy.

Alternatively, with respect to a N^{th} piezoelectric element, . . . , a third piezoelectric element, and a second piezoelectric element (wherein N is 1, 2, 3, . . . ) which are arranged to be spaced apart from one another in sequential order in the counterclockwise direction based on a first piezoelectric element, the control unit controls energy injected using any one among frequency (MHz), pressure (w/cm²), volt (V), and relay time of the ultrasound generator as an independent variable to satisfy the sequential order of the N^{th} piezoelectric element, . . . , the third piezoelectric element, the second piezoelectric element, and the first piezoelectric element, thereby continuously forming counterclockwise ultrasound vortex in sequential order of a body area receiving ultrasound waves from the N^{th} piezoelectric element, . . . , a body area receiving ultrasound waves from the third piezoelectric element, a body area receiving ultrasound waves from the second piezoelectric element, and a body area receiving ultrasound waves from the first piezoelectric element due to a difference of thermal storage energy in proportion to injected energy.

As described above, according to a preferred embodiment of the present invention, the ultrasound vortex energy injection device has three or more piezoelectric elements arranged at equal intervals in the circumferential direction of the inclined area formed on the outer circumference of the vacuum unit of the lower housing to disperse and inject ultrasound waves, thereby enabling more effective stimulation and treatment due to overlap and diffraction between ultrasound waves transmitted from the piezoelectric elements, and preventing the thermal island phenomenon caused by the conventional ultrasound focusing.

Moreover, with respect to the three or more piezoelectric elements arranged in the circumferential direction of the inclined area formed on the outer circumference of the vacuum unit of the lower housing, the ultrasound vortex energy injection device according to the present invention can control the energy injected from the ultrasound generator to satisfy the sequential order of the first piezoelectric element, the second piezoelectric element, the third piezoelectric element, . . . , and the N^{th} piezoelectric element, thereby continuously forming clockwise ultrasound vortex in sequential order of a body area receiving ultrasound waves from the first piezoelectric element, a body area receiving ultrasound waves from the second piezoelectric element, a body area receiving ultrasound waves from the third piezoelectric element, . . . , and a body area receiving ultrasound waves from the N^{th} piezoelectric element due to a difference of thermal storage energy in proportion to injected energy. Alternatively, the ultrasound vortex energy injection device according to the present invention can control the energy to satisfy the sequential order of the N^{th} piezoelectric element, . . . , the third piezoelectric element, the second piezoelectric element, . . . , and the first piezoelectric element, thereby continuously forming counterclockwise ultrasound vortex in sequential order of a body area receiving ultrasound waves from the N^{th} piezoelectric element, . . . , a body area receiving ultrasound waves from the third piezoelectric element, a body area receiving ultrasound waves from the second piezoelectric element, and a body area receiving ultrasound waves from the first piezoelectric element due to a difference of thermal storage energy in proportion to injected energy.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is an exploded perspective view of an ultrasound vortex energy injection device according to a preferred embodiment of the present invention;

FIG. 2 is an exploded sectional view of the ultrasound vortex energy injection device according to the preferred embodiment of the present invention; and

FIG. 3 is a plan view illustrating a connector of the ultrasound vortex energy injection device according to the preferred embodiment of the present invention.

### DETAILED DESCRIPTION OF THE PREFERRED EMBODIMENT

Hereinafter, the present disclosure will be described in more detail with reference to the exemplary embodiments. However, the present invention is not limited to exemplary embodiment disclosed herein but will be implemented in various forms. The exemplary embodiments are provided so that the present invention is completely disclosed, and a person of ordinary skilled in the art can fully understand the scope of the present invention. In the drawings, the same reference numerals will be used throughout to designate the same or like components.

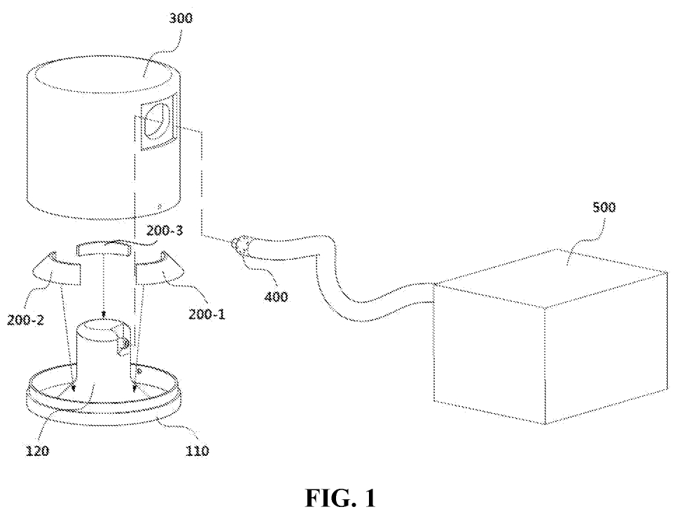

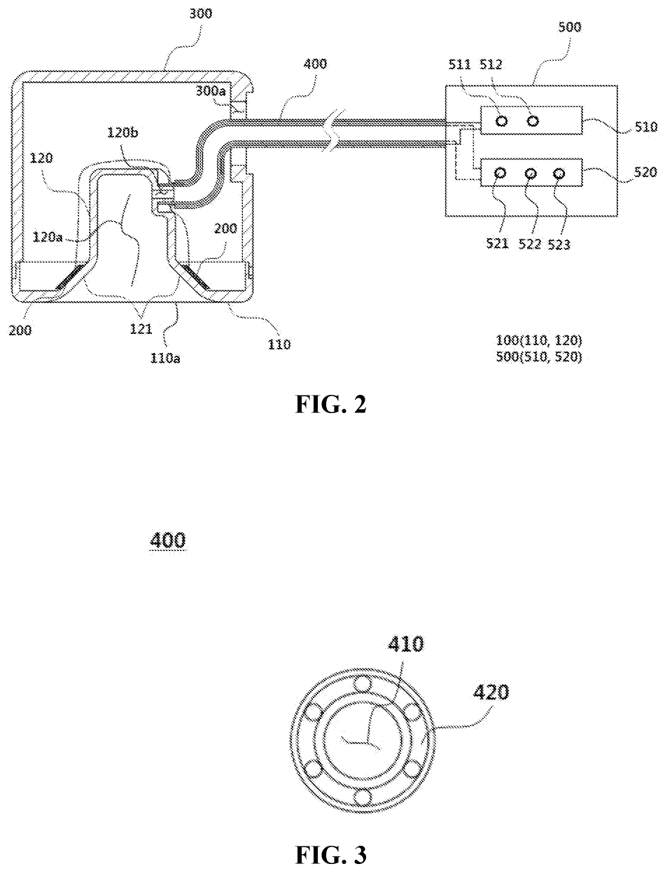

FIG. 1 is an exploded perspective view of an ultrasound vortex energy injection device according to a preferred embodiment of the present invention, FIG. 2 is an exploded sectional view of the ultrasound vortex energy injection device according to the preferred embodiment of the present invention, and FIG. 3 is a plan view illustrating a connector of the ultrasound vortex energy injection device according to the preferred embodiment of the present invention.

As illustrated in FIGS. 1 to 3, the present invention relates to an ultrasound vortex energy injection device and, more specifically, to an ultrasound vortex energy injection device configured in such a way as to disperse ultrasound waves without focusing the ultrasound waves with respect to an area coming in contact with a human body, and at the same time, apply a vortex-type stimulus in a circumferential direction of an area getting in contact with the human body with respect to a plurality of piezoelectric elements arranged to be spaced apart from one another in a circumferential direction (clockwise or counterclockwise) of an area opposed to the human body where ultrasound waves are transmitted. The ultrasound vortex energy injection device includes: a lower housing 100, which has a doughnut-shaped ultrasound transducer 110 having a vacuum hole 110a perforated at the center, and a trumpet-shaped vacuum unit 120 protruding and extending upward from the vacuum hole 110a of the ultrasound transducer 110 and having a vacuum groove 120a; piezoelectric elements 200 arranged on the outer circumference of an inclined area 121 of which the circumference is gradually increased, of the area of the vacuum unit 120 having the vacuum groove 120a; an upper housing 300 coupled to the lower housing 100; a connector 400 connected to the vacuum groove 120a and the piezoelectric elements 200 through the upper housing 300; and a control unit 500 connected to the connector 400 to form negative pressure in an inner space of the vacuum groove 120a, and controlling ultrasound waves to be generated in the piezoelectric elements 200.

The lower housing 100 is a means for forming an inner space to make a negative pressure state by getting in contact with the outer circumferential surface of an area to be stimulated, among body parts of a user. The lower housing 100 includes: a doughnut-shaped ultrasound transducer 110 having a vacuum hole 110a perforated at the center, and a vacuum unit 120 protruding and extending upward from the vacuum hole 110a of the ultrasound transducer 110; and a vacuum unit 120 protruding upward from the outer circumferential surface of the vacuum hole 110a of the ultrasound transducer 110 and having a vacuum groove 120a opened at a lower portion and a connector connection hole 120b formed at one side of the outer circumferential surface of the protruding upper portion and communicating with the inside of the vacuum groove 120a.

Especially, the outer circumferential surface of an area in which the vacuum groove 120a of the vacuum unit 120 is formed is formed in a trumpet shape gradually increasing downward.

The piezoelectric elements 200 convert electrical vibration into mechanical vibration to transfer the converted mechanical vibration to an area to be stimulated among the user's body parts. A lower area of the area in which the vacuum groove 120a of the ultrasound transducer 110 is formed has an inclined area 121 in which the circumference gradually increases toward the lower end, and the piezoelectric elements 200 are arranged at equal intervals in the circumferential direction of the outer circumference of the inclined area 121.

In particular, the piezoelectric elements 200 of the present invention are arranged at three or more intervals in the circumferential direction of the outer circumference of the inclined region 121.

Accordingly, the mechanical vibration generated from the plurality of piezoelectric elements 200 can be transmitted to an area to be stimulated by the mutual overlap/diffraction, thereby preventing thermal runaway due to the ultrasound focusing.

The upper housing 300 is a cover covering the lower housing 100. The upper housing 300 is formed in a cylindrical shape having a hollow inside and an open lower surface, and is detachably combined with the outer circumferential surface of the ultrasound transducer 110 of the lower housing 100. A connector insertion hole 300a is formed at one side of the outer circumferential surface of the upper end of the upper housing 300.

The connector 400 includes: a central portion 410 penetrating the connector insertion hole 300a to communicate with the connector connection hole 120b; and a circumferential portion 420 provided with a cable of an ultrasound generator electrically connected to the piezoelectric elements 200, the cable being disposed around the outer circumferential surface of the central portion 410.

The central portion 410 is electrically connected to the vacuum generator 510 to form negative pressure in the inner region of the vacuum groove 120a, and one end of the cable provided in the circumferential direction of the circumferential portion 420 is electrically connected to the ultrasound generator 520, and the other end of the cable is connected to the piezoelectric elements 200.

The control unit 500 is a means for controlling the operation of the vacuum generator 510 and the ultrasound generator 520. The control unit 500 includes the vacuum generator 510 and the ultrasound generator 520 therein, wherein the vacuum generator 510 is connected to the central portion 410 of the connector 400 by means of a hose, and the ultrasound generator 520 is electrically connected to the cable of the ultrasound generator.

In particular, the control unit 500 includes: a power button 511 for turning on/off the operation of the vacuum generator 510; a vacuum control button 512 for adjusting a pulse value of the vacuum generator 510; a power button 521 for turning on/off the operation of the ultrasound generator 520; an ultrasound control button 522 for controlling the ultrasound frequency and pressure of the ultrasound generator 520; and a left/right torsion setting button 530.

The frequency of the ultrasound waves generated by the ultrasound generator 520 ranges from 0.5 to 1.5 MHz, pressure ranges from 0.5 to 3 W/cm², and the relay time ranges from 10 to 20 mSec(ON)/from 1 to 2 mSec(OFF).

The left/right torsion setting button 530 can set left torsion when being pressurized once, and set right torsion when being continuously pressurized twice. It is preferable that the set torsion operation continues for three minutes or more.

In particular, with respect to a second piezoelectric element 200-2, a third piezoelectric element 200-3, . . . , and a N^{th} piezoelectric element 200-3+N . . . (wherein N is 1, 2, 3, . . . ) which are arranged to be spaced apart from one another in sequential order in the clockwise direction based on a first piezoelectric element 200-1, the control unit 500 controls energy injected using any one among frequency (MHz), pressure (w/cm²), volt (V), and relay time of the ultrasound generator 520 as an independent variable to satisfy the sequential order of the first piezoelectric element 200-1, the second piezoelectric element 200-2, the third piezoelectric element 200-3, . . . , and the N^{th} piezoelectric element 200-3+N . . . , thereby continuously forming clockwise ultrasound vortex in sequential order of a body area receiving ultrasound waves from the first piezoelectric element 200-1, a body area receiving ultrasound waves from the second piezoelectric element 200-2, and a body area receiving ultrasound waves from the third piezoelectric element 200-3 due to a difference of thermal storage energy in proportion to injected energy.

In the following, Table 1 is an example in which clockwise ultrasound vortex is formed using frequency as an independent variable, Table 2 is an example in which clockwise ultrasound vortex is formed using pressure as an independent variable, Table 3 is an example in which clockwise ultrasound vortex is formed using volt as an independent variable, and Table 4 is an example in which clockwise ultrasound vortex is formed using relay time as an independent variable.

TABLE 1

| | | | | Relay | | |
|---|---|---|---|---|---|---|
| | Frequency | Pressure | Volt | Time(mSec) | | Energy |
| Example 1 | (MHz) | (w/cm²) | (V) | ON | OFF | (Kcal) |
| Piezoelectric | 1.2 | 1.0 | 80 | 15 | 2 | 300 |
| element 200-1 | | | | | | |
| Piezoelectric | 1.0 | 1.0 | 80 | 15 | 2 | 200 |
| element 200-2 | | | | | | |
| Piezoelectric | 0.8 | 1.0 | 80 | 15 | 2 | 100 |
| element 200-3 | | | | | | |

TABLE 2

| | | | | Relay | | |
|---|---|---|---|---|---|---|
| | Frequency | Pressure | Volt | Time(mSec) | | Energy |
| Example 1 | (MHz) | (w/cm²) | (V) | ON | OFF | (Kcal) |
| Piezoelectric | 1.0 | 1.5 | 80 | 15 | 2 | 300 |
| element 200-1 | | | | | | |
| Piezoelectric | 1.0 | 1.0 | 80 | 15 | 2 | 200 |
| element 200-2 | | | | | | |
| Piezoelectric | 1.0 | 0.5 | 80 | 15 | 2 | 100 |
| element 200-3 | | | | | | |

TABLE 3

| | | | | Relay | | |
|---|---|---|---|---|---|---|
| | Frequency | Pressure | Volt | Time(mSec) | | Energy |
| Example 1 | (MHz) | (w/cm²) | (V) | ON | OFF | (Kcal) |
| Piezoelectric | 1.0 | 1.0 | 85 | 15 | 2 | 300 |
| element 200-1 | | | | | | |
| Piezoelectric | 1.0 | 1.0 | 80 | 15 | 2 | 200 |
| element 200-2 | | | | | | |
| Piezoelectric | 1.0 | 1.0 | 75 | 15 | 2 | 100 |
| element 200-3 | | | | | | |

TABLE 4

| | | | | Relay | | |
|---|---|---|---|---|---|---|
| | Frequency | Pressure | Volt | Time(mSec) | | Energy |
| Example 4 | (MHz) | (w/cm²) | (V) | ON | OFF | (Kcal) |
| Piezoelectric | 1.0 | 1.0 | 80 | 20 | 2 | 300 |
| element 200-1 | | | | | | |
| Piezoelectric | 1.0 | 1.0 | 80 | 15 | 2 | 200 |
| element 200-2 | | | | | | |
| Piezoelectric | 1.0 | 1.0 | 80 | 10 | 2 | 100 |
| element 200-3 | | | | | | |

As shown in Examples 1 to 4, when the ultrasound generator 520 controls energy injected into the piezoelectric elements 200 using any one among frequency, pressure, volt, and relay time as an independent variable to satisfy the sequential order of the first piezoelectric element 200-1, the second piezoelectric element 200-2, and the third piezoelectric element 200-3, clockwise ultrasound vortex is continuously formed in sequential order of a body area receiving ultrasound waves from the first piezoelectric element 200-1, a body area receiving ultrasound waves from the second piezoelectric element 200-2, and a body area receiving ultrasound waves from the third piezoelectric element 200-3 due to a difference of thermal storage energy in proportion to injected energy.

Furthermore, with respect to a N^{th} piezoelectric element 200-3+N, . . . , a third piezoelectric element 200-3, and a second piezoelectric element 200-2 (wherein N is 1, 2, 3, . . . ) which are arranged to be spaced apart from one another in sequential order in the counterclockwise direction based on a first piezoelectric element 200-1, the control unit 500 controls energy injected using any one among frequency (MHz), pressure (w/cm²), volt (V), and relay time of the ultrasound generator 520 as an independent variable to satisfy the sequential order of the third piezoelectric element 200-3, the second piezoelectric element 200-2, and the first piezoelectric element 200-1, thereby continuously forming clockwise ultrasound vortex in sequential order of a body area receiving ultrasound waves from the third piezoelectric element 200-3, a body area receiving ultrasound waves from the second piezoelectric element 200-2, and a body area receiving ultrasound waves from the first piezoelectric element 200-1 due to a difference of thermal storage energy in proportion to injected energy.

In the following, Table 5 is an example in which clockwise ultrasound vortex is formed using frequency as an independent variable, Table 6 is an example in which clockwise ultrasound vortex is formed using pressure as an independent variable, Table 7 is an example in which clockwise ultrasound vortex is formed using volt as an independent variable, and Table 8 is an example in which clockwise ultrasound vortex is formed using relay time as an independent variable.

TABLE 5

| | | | | Relay | | |
|---|---|---|---|---|---|---|
| | Frequency | Pressure | Volt | Time(mSec) | | Energy |
| Example 1 | (MHz) | (w/cm²) | (V) | ON | OFF | (Kcal) |
| Piezoelectric | 0.8 | 1.0 | 80 | 15 | 2 | 300 |
| element 200-1 | | | | | | |
| Piezoelectric | 1.0 | 1.0 | 80 | 15 | 2 | 200 |
| element 200-2 | | | | | | |
| Piezoelectric | 1.2 | 1.0 | 80 | 15 | 2 | 100 |
| element 200-3 | | | | | | |

TABLE 6

| | | | | Relay | | |
|---|---|---|---|---|---|---|
| | Frequency | Pressure | Volt | Time(mSec) | | Energy |
| Example 1 | (MHz) | (w/cm²) | (V) | ON | OFF | (Kcal) |
| Piezoelectric | 1.0 | 0.5 | 80 | 15 | 2 | 300 |
| element 200-1 | | | | | | |
| Piezoelectric | 1.0 | 1.0 | 80 | 15 | 2 | 200 |
| element 200-2 | | | | | | |
| Piezoelectric | 1.0 | 1.5 | 80 | 15 | 2 | 100 |
| element 200-3 | | | | | | |

TABLE 7

| | | | | Relay | | |
|---|---|---|---|---|---|---|
| | Frequency | Pressure | Volt | Time(mSec) | | Energy |
| Example 1 | (MHz) | (w/cm²) | (V) | ON | OFF | (Kcal) |
| Piezoelectric | 1.0 | 1.0 | 75 | 15 | 2 | 300 |
| element 200-1 | | | | | | |
| Piezoelectric | 1.0 | 1.0 | 80 | 15 | 2 | 200 |
| element 200-2 | | | | | | |
| Piezoelectric | 1.0 | 1.0 | 85 | 15 | 2 | 100 |
| element 200-3 | | | | | | |

TABLE 8

| | | | | Relay | | |
|---|---|---|---|---|---|---|
| | Frequency | Pressure | Volt | Time(mSec) | | Energy |
| Example 4 | (MHz) | (w/cm²) | (V) | ON | OFF | (Kcal) |
| Piezoelectric | 1.0 | 1.0 | 80 | 10 | 2 | 300 |
| element 200-1 | | | | | | |
| Piezoelectric | 1.0 | 1.0 | 80 | 15 | 2 | 200 |
| element 200-2 | | | | | | |
| Piezoelectric | 1.0 | 1.0 | 80 | 20 | 2 | 100 |
| element 200-3 | | | | | | |

As shown in Examples 5 to 8, when the ultrasound generator 520 controls energy injected into the piezoelectric elements 200 using any one among frequency, pressure, volt, and relay time as an independent variable to satisfy the sequential order of the third piezoelectric element 200-3, the second piezoelectric element 200-2, and the first piezoelectric element 200-1, clockwise ultrasound vortex is continuously formed in sequential order of a body area receiving ultrasound waves from the first piezoelectric element 200-1, a body area receiving ultrasound waves from the second piezoelectric element 200-2, and a body area receiving ultrasound waves from the third piezoelectric element 200-3 due to a difference of thermal storage energy in proportion to injected energy.

In other words, the ultrasound vortex energy injection device according to the present invention controls energy injected in the ultrasound generator with respect to the three or more piezoelectric elements arranged in the circumferential direction of the inclined area formed on the outer circumference of the vacuum unit of the lower housing to satisfy the sequential order of the first piezoelectric element 200-1, the second piezoelectric element 200-2, the third piezoelectric element 200-3, . . . , and the N^{th} piezoelectric element 200-3+N . . . or the sequential order of the first piezoelectric element 200-1, the N^{th} piezoelectric element 200-3+N, . . . , the third piezoelectric element 200-3, and the second piezoelectric element 200-2, thereby sequentially transferring energy from an area in which high energy is stored by the piezoelectric element injecting high energy to an area receiving energy by the piezoelectric element in which energy with a relatively low level is stored.

Meanwhile, in the case where the relay time is used as an independent variable, the ON-state of the relay time is used as the independent variable, but the present invention is not limited thereto and the OFF-state of the relay time can be used as the independent variable.

Although the exemplary embodiments of the present invention have been disclosed for illustrative purposes, the present invention is not limited to the exemplary embodiments described herein and will be limited by the accompanying claims. Therefore, it will be understood by those of ordinary skill in the art that various modifications and equivalents may be made without deviating from the spirit or scope of the invention.

What is claimed is:

1. An ultrasound vortex energy injection device comprising:
a lower housing, which has a doughnut-shaped ultrasound transducer having a vacuum hole perforated at the center, and a trumpet-shaped vacuum unit protruding and extending upward from the vacuum hole of the ultrasound transducer and having a vacuum groove;
three or more piezoelectric elements arranged on the outer circumference of an inclined area which is formed at a lower area of the area in which the vacuum groove of the vacuum unit is formed, and of which the circumference is gradually increased, the three or more piezoelectric elements being arranged on the inclined area at equal intervals;
an upper housing connected to the outer circumferential surface of the ultrasound transducer of the lower housing and having a connector insertion hole formed at one side of the outer circumferential surface of the upper end;
a connector which has a central portion penetrating the connector insertion hole to communicate with the connector connection hole, and a circumferential portion provided with a cable of an ultrasound generator electrically connected to the piezoelectric elements, the cable being disposed around the outer circumferential surface of the central portion; and
a control unit which includes a vacuum generator connected to the central portion of the connector by means of a hose, and an ultrasound generator electrically connected to the cable of the ultrasound generator so as to control the operation of the vacuum generator and the ultrasound generator.

2. The ultrasound vortex energy injection device according to claim 1, wherein with respect to a second piezoelectric element, a third piezoelectric element, . . . , and a N^{th} piezoelectric element (wherein N is 1, 2, 3, . . . ) which are arranged to be spaced apart from one another in sequential order in the clockwise direction based on a first piezoelectric element, the control unit controls energy injected using any one among frequency (MHz), pressure (w/cm²), volt (V), and relay time of the ultrasound generator as an independent variable to satisfy the sequential order of the first piezoelectric element, the second piezoelectric element, the third piezoelectric element, . . . , and the N^{th} piezoelectric element.

3. The ultrasound vortex energy injection device according to claim 1, wherein with respect to a N^{th} piezoelectric element, . . . , a third piezoelectric element, and a second piezoelectric element (wherein N is 1, 2, 3, . . . ) which are arranged to be spaced apart from one another in sequential order in the counterclockwise direction based on a first piezoelectric element, the control unit controls energy injected using any one among frequency (MHz), pressure (w/cm²), volt (V), and relay time of the ultrasound generator as an independent variable to satisfy the sequential order of the N^{th} piezoelectric element, . . . , the third piezoelectric element, the second piezoelectric element, and the first piezoelectric element.
